SEQUENCE LISTING (28 sequences)

SEQ ID NO: 1 - protein, 378 aa, synthetic construct
LQLDASTVYAVGRGDSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRGDSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRGDSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRGDSPASSAVPGIGVPGIGVPGKGVPGIGVPGIGVPGIGVPGIGVPGKGVPGIGVPGIGVPGIGVPGIGVPGKGVPGIGVPGIG

SEQ ID NO: 2 - protein, 378 aa, synthetic construct
LQLDASTVYAVGRDGSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRDGSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRDGSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRDGSPASSAVPGIGVPGIGVPGKGVPGIGVPGIGVPGIGVPGIGVPGKGVPGIGVPGIGVPGIGVPGIGVPGKGVPGIGVPGIG

SEQ ID NO: 3 - protein, 400 aa, synthetic construct
MASMTGGQQHHHHHHDDDDKLQLDASTVYAVGRXXSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRXXSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRXXSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRXXSPASSAVPGIGVPGIGVPGKGVPGIGVPGIGVPGIGVPGIGVPGKGVPGIGVPGIGVPGIGVPGIGVPGKGVPGIGVPGIGLE

SEQ ID NO: 4 - protein, 12 aa, synthetic construct
PLAEIDGIELTY

SEQ ID NO: 5 - protein, 8 aa, synthetic construct
VFDNFVLK

SEQ ID NO: 6 - protein, 5 aa, synthetic construct
IKVAV

SEQ ID NO: 7 - protein, 5 aa, synthetic construct
YIGSR

SEQ ID NO: 8 - protein, 5 aa, synthetic construct
HAVDI

SEQ ID NO: 9 - protein, 10 aa, synthetic construct
HAVDIHAVDI

SEQ ID NO: 10 - protein, 17 aa, synthetic construct
TVYAVTGRGDSPASSAA

SEQ ID NO: 11 - protein, 20 aa, synthetic construct
VSDPGYIGSRSDDSASASAA

SEQ ID NO: 12 - protein, 16 aa, synthetic construct
VGPAGGDGEAGAQGPP

SEQ ID NO: 13 - protein, 28 aa, synthetic construct
SGSGGSGGPLAEIDGIELTYGGSGGSGS

SEQ ID NO: 14 - protein, 26 aa, synthetic construct
SGSGGSGGLDVFDNFVLKGGSGGSGS

SEQ ID NO: 15 - protein, 25 aa, synthetic construct
SGSGGSGGLDVFDNFVLGGSGGSGS

SEQ ID NO: 16 - protein, 21 aa, synthetic construct
SGSGGSGGHAVDIGGSGGSGS

SEQ ID NO: 17 - protein, 28 aa, synthetic construct
SGSGGSGGHAVDINGHAVDIGGSGGSGS

SEQ ID NO: 18 - protein, 21 aa, synthetic construct
SGSGGSGGADHIVGGSGGSGS

SEQ ID NO: 19 - protein, 17 aa, synthetic construct
TVYAVTGRDGSPASSAA

SEQ ID NO: 20 - protein, 25 aa, synthetic construct
VPGIGVPGIGVPGKGVPGIGVPGIG

SEQ ID NO: 21 - protein, 30 aa, synthetic construct
VPGIGVPGKGVPGYGVPGIGVPGKGVPGIG

SEQ ID NO: 22 - protein, 19 aa, synthetic construct
ARKQAASIKVAVSADRASA

SEQ ID NO: 23 - protein, 5 aa, synthetic construct
VPGIG

SEQ ID NO: 24 - protein, 5 aa, synthetic construct
VPGKG

SEQ ID NO: 25 - protein, 5 aa, synthetic construct
VPGYG

SEQ ID NO: 26 - protein, 4 aa, synthetic construct
DGEA

SEQ ID NO: 27 - protein, 5 aa, unidentified
GGGGS

SEQ ID NO: 28 - protein, 14 aa, synthetic construct
GSTSGSGKSSEGKG

(19) United States
(12) Patent Application Publication
(10) Pub. No.: US 2024/0366501 A1
(43) Pub. Date: Nov. 7, 2024

(54) HYDROGEL INJECTION FOR INTESTINAL ANCHORING

(71) Applicant: The Board of Trustees of the Leland Stanford Junior University, Stanford, CA (US)

(72) Inventors: James Chungyu Dunn, Stanford, CA (US); Sarah C. Heilshorn, Mountain View, CA (US); Fereshteh Salimi-Jazi, Redwood City, CA (US); Anne-Laure Thomas, Redwood City, CA (US); Narelli De Paiva Narciso, Stanford, CA (US); Riley Suhar, San Leandro, CA (US); Renato Navarro, Menlo Park, CA (US)

(21) Appl. No.: 18/653,524

(22) Filed: May 2, 2024

Related U.S. Application Data
(60) Provisional application No. 63/463,782, filed on May 3, 2023.

(51) Int. Cl. A61K 9/00; A61K 47/34; A61M 31/00

(52) U.S. Cl. CPC A61K 9/0024; A61K 47/34; A61M 31/007

(57) ABSTRACT

Compositions and methods are provided for the use of hydrogel compositions to anchor a device, or therapeutic entity, to a specific portion of the GI tract. In some embodiments a biodegradable hydrogel is injected into the intestinal wall to narrow the lumen, such that a device can be confined to that specific segment of the intestine for a desired period of time. In some embodiments the device is an in situ expander for distraction enterogenesis of the intestine.

### CROSS-REFERENCE TO RELATED APPLICATIONS

This application claims the benefit of U.S. Provisional Patent Application No. 63/463,782, filed May 3, 2023, which application is incorporated herein by reference in its entirety.

### INCORPORATION BY REFERENCE OF SEQUENCE LISTING

A Sequence Listing is provided herewith as a Sequence Listing XML, “STAN-2100_Sequence_Listing created on Apr. 15, 2024, and having a size of 26,360 bytes. The contents of the Sequence Listing XML are incorporated herein by reference in their entirety.

### BACKGROUND

Short bowel syndrome (SBS) is a devastating malabsorptive condition associated with a lack of functional intestinal surface area of bowel. Due to this lack of absorptive surface area, the remnant intestine cannot adequately absorb nutrients, resulting in the inability of the enteral source of nutrition to sustain growth, development and life. Complications from SBS frequently result from medical therapies to support this condition. SBS may result from congenital or acquired conditions. Mostly, SBS results from extensive surgical resection of the small intestine due to midgut volvulus, malrotation, extensive aganglionosis or necrotizing enterocolitis, the latter reflecting the most common cause of bowel resection in children (29% of cases). In adults, multiple small bowel resections related to recurrent Crohn's disease or massive resections due to catastrophic mesenteric events (arterial embolism, venous thrombosis), trauma or malignancies represent the most common causes of SBS.

Different approaches have been investigated as treatments for SBS, including supportive measures such as parenteral nutrition, medications to increase intestinal absorptive capacity by slowing transit and invasive surgical lengthening procedures such as serial transverse enteroplasty. However, these approaches have relatively high morbidity and mortality rates, and their complications are costly to manage compared to the level of success to achieve enteral autonomy.

Generating novel therapies may obviate the need for the current methods of treatments and their high complication rates in patients with SBS. Mechanical stimulus has been extensively examined as an important factor in stimulating tissue growth and proliferation, where distraction enterogenesis can increase intestinal length and absorptive surface area. Distraction enterogenesis relies on applying a mechanical force to a segment of intestine to promote intestinal lengthening; a variety of methods and device considerations have been investigated to incorporate the external mechanical force to the distracted segment of bowel.

The ultimate goal of SBS treatment is to achieve full intestinal autonomy and to reduce long-term dependence on parenteral support by increasing the absorptive capacity of the remnant bowel. Methods of improving long term therapies are of great clinical interest.

### Publications

Stark and Dunn (2012) J. Healthcare Engineering 3 (2): 229-242 review mechanical enterogenesis. A feasibility study to evaluate safety & benefit of the Eclipse XL1 system in adult patients with SBS is disclosed in Clinical Trial NCT05535361.

Portelli et al. (2022) Journal of Pediatric Surgery VOLUME 57, ISSUE 7, P1377-1381 relates to distraction enterogenesis in the murine colon. Hosseini et al. (2020) Mechanical Behavior of Biomedical Materials 101:103413 disclose biomechanics of small intestine during distraction enterogenesis with an intraluminal spring.

Hydrogel compositions and methods of use are described in PCT publication WO 2022/098777, herein specifically incorporated by reference.

### SUMMARY

Compositions and methods are provided for the use of hydrogel compositions to anchor a device, or therapeutic entity, to a specific portion of the GI tract. In some embodiments a biodegradable hydrogel is injected into the intestinal wall to narrow the lumen, such that a device can be confined to that specific segment of the intestine for a desired period of time. In some embodiments the device is an in situ expander for distraction enterogenesis of the intestine. In some embodiments the in situ expander is a compressed coiled spring. In some embodiments the individual is suffering from short bowel syndrome. The hydrogel is injected at a dose effective to anchor the device or therapeutic entity in the intestine.

A hydrogel composition of the disclosure comprises: (1) chemically modified hyaluronic acid (HA), and (2) chemically modified elastin-like protein (ELP). Mixing the two modified biopolymers together induces the formation of hydrazone bonds resulting in a hydrogel network, referred to herein as “HELP”. Selection of the ratio between HA and ELP, and the ratio of variants of these components allows tuning of critical variables of HELP, including, for example, matrix stiffness, matrix stress relaxation rate, and cell-adhesive-ligand concentration and identity. These variables can be independently and quantitatively defined. The hyaluronic acid component is chemically modified to comprise a pendant benzaldehyde or aldehyde side group. The specific ratio of these two chemical groups in the final hydrogel formulation controls the stress relaxation variable. The ratio of HA-benzaldehyde to HA-aldehyde may be pre-selected for a hydrogel of interest, usually ranging from about 100:0 to 0:100, for example at a ratio from about 95:5, 90:10, 75:25, 50:50, 25:75, etc.

In some embodiments, the hydrogel for intestinal anchoring comprises hyaluronic acid where at least about 6% up to about 20%, e.g. at least about 8%, at least about 10%, at least about 12%, up to about 18%, up to about 16%, up to about 15% is conjugated with aldehyde; and where at least about 6% up to about 30%, e.g. at least about 10%, at least about 15%, at least about 20%, up to about 30%, up to about 25% is conjugated with aldehyde.

The ELP component of the hydrogel comprises a recombinant sequence of elastin-like sequences optionally interspersed with cell-adhesive sequences. Examples of ELP sequences are provided in SEQ ID NO:1, 2 and 3, where SEQ ID NO:1 and SEQ ID NO:2 are ELPs with an RGD sequence, and a scrambled RGD sequence, and SEQ ID NO:3 is an ELP amino acid sequence that can be modified to contain cell-adhesive motifs. To engage in crosslinking with chemically modified HA, the ELP is chemically modified to comprise one or more pendant hydrazine groups. The optional cell-adhesive sequence within the ELP may be selected from an integrin-binding, fibronectin-based, extended RGD sequence, a scrambled RGD sequence, a cell-adhesive sequence derived from collagen type I, e.g. (SEQ ID NO:26) DGEA, a cell adhesive sequence derived from tenascin, e.g. (SEQ ID NO:4) PLAEIDGIELTY, (SEQ ID NO:5) VFDNFVLK, etc.; a cell adhesive sequence derived from laminin, e.g. (SEQ ID NO:6) IKVAV, (SEQ ID NO:7) YIGSR, etc.; a cell adhesive sequence derived from cadherin, e.g. (SEQ ID NO:8) HAVDI, (SEQ ID NO:9) HAVDIHAVDI; and the like.

In some embodiments the ELP protein for intestinal anchoring is chemically modified with a pendant hydrazine group comprising from about 3 to about 20 hydrazine groups, from about 5 to about 18, from about 10 to about 14 groups.

Matrix stiffness is determined by the concentration and ratio of hydrazine to aldehyde and benzaldehyde reactive groups, where a ratio of about 1:1 provides for maximum cross-linking. The ratio may be varied, e.g. from about 1:3, about 1:2, about 1.5:1, about 1.25:1, about 1:1, about 1:1.25, about 1:1.5, about 1:2, about 1:3, etc.

In some embodiments the ratio of ELP to HA in a hydrogel for intestinal anchoring is from about 1.5:1 to about 1:1.5 by weight; and may be about 1.75:1 to about 1:1.75 In some embodiments the ratio is about 1:1.

In an embodiment of the invention, the matrix is extruded through a syringe needle or a catheter to the intestinal site for anchoring. After extrusion, the HELP matrix reforms a gel-phase material. The overall polymer concentration may have a final concentration of from about 0.5-1.5 wt % of ELP, and may be about 1% ELP; and from about 0.5-1.5 wt % of HA and may be about 1% HA. The effective dose will be determined by the diameter of the intestine, and may be from 1 to about 50 ml. per application, e.g. from about 1 to 40, 1 to 30, 1 to 20, 1 to 10, 1-5 ml.

In am embodiments, an effective amount of the hydrogel is injected into the submucosa of the intestine proximal and distal to an in situ expander for distraction enterogenesis of the intestine partially occlude the lumen and keep the in situ expander in place. In some embodiments the in situ expander is an encapsulated compressed nitinol spring.

### BRIEF DESCRIPTION OF THE DRAWINGS

FIG. 1. In vitro study: Hydrogel was injected into the submucosa of the retrieved intestine to hold the spring. The specimen was formalin fixed for 48 hours, cross-sectioned, and stained with H&E. Slides were imaged under bright field microscopy.

FIG. 2A-2B. In vivo study: 6 Juvenile pigs were used for the study. After enterotomy, transected jejunum was everted and 1.5 to 4 ml of HA-hydrogel was injected into the submucosa of the intestine proximal and distal to the gelatin-encapsulated compressed nitinol spring to partially occlude the lumen and keep the spring in place. Non-absorbable sutures were placed on the serosa of control and spring-containing intestinal segments for length comparison. Pigs were placed on clear liquid diet and euthanized on POD7. Intestinal segments were evaluated for lengthening between marking sutures and processed for histologic examination.

FIG. 3A-3B. Lengthening results.

FIG. 4. Histology results.

FIG. 5A-5B. Histology results.

### DETAILED DESCRIPTION

### Definitions

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a”, “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

Short bowel syndrome (SBS) is a highly morbid disorder caused by inadequate length of functional small intestine. It is characterized by malnutrition, malabsorption and dehydration. Although the diagnosis is both clinical and highly variable, it is commonly associated with the loss of 70% or more of small intestinal length. Common causes of SBS in the pediatric population are necrotizing enterocolitis, aganglionosis, intestinal atresias, midgut volvulus, and abdominal wall defects. In the adult population, the most common cause is surgical resection for trauma or intestinal ischemia. Mortality rate of SBS in the pediatric population approaches 30% and the surviving children face a barrage of morbidities.

Current management of SBS includes supportive therapies, hormone administration, bowel lengthening surgeries, and small bowel transplantation. Supportive therapies include parenteral nutrition, anti-motility agents, ursodiol, and vitamin supplementation. Surgical therapies for SBS involve interposition of reversed intestine, longitudinal intestinal lengthening and tailoring and serial transverse enteroplasty procedure (STEP). All these techniques have varying results and are rarely curative. Currently, the final treatment for SBS is small bowel transplantation; problems thereof include five-year survival rates of approximately 50%, the requirement of lifelong immune suppression and the paucity of available donors.

Mechanical enterogenesis is a method of lengthening pre-existing intestine with distractive force. The application of distractive forces on the small intestine aims to induce cellular proliferation and ultimately increase bowel length. This has been investigated primarily for the treatment of short bowel syndrome (SBS). The underlying principle of mechanical enterogenesis is that the increased surface area achieved by mechanical strain results from new cellular regeneration or growth and is not secondary to deformations from stretch. This so called mechanotransduction involves a multitude of signaling cascades and chemical pathways that ultimately result in cellular proliferation. For example, see U.S. Pat. No. 9,138,336, herein specifically incorporated by reference.

Several reports have shown successful lengthening of intestine in both rats and pigs with preservation of the mechanical properties of intestinal smooth muscle as well as enzymatic and absorptive properties. Stretched intestine has been shown to contract similarly to stimulation as non-stretched intestine. Several studies have measured enzymatic activity and absorptive capacity in stretched small bowel with encouraging results. Results suggest that there is little to no difference between the stretched and non-stretched intestine in relation to barrier function and absorptive capacity.

A challenge in creating a mechanism for stretching intestinal tissue is designing an in situ expander. Ideally, an expander would be placed within the lumen of small intestine in continuity with the entire bowel. Current models have focused on surgically placing expanding devices within segments of isolated small intestine, which after expansion would be reinstated into continuity with the remaining intestine. Drawbacks to this method include the additional operation as well as the length of bowel lost with re-anastamosis.

In some embodiments of the disclosure, an expandable mechanical distension device is applied for expansion of the small intestine. Included for this purpose is a medical device using biomaterials including nickel titanium, stainless steel, or bio-degradable shape memory polymer, that elongates by mechanical expansion. The device preferably expands radially to engage the internal walls of the intestine at the desired location and expands axially to enlarge the intestine segment. The distension device may be configured for the treatment of patients with insufficient hollow viscus, e.g. short gut syndrome, to enhance their gut length by mechanical force.

The mechanical distension device may be made of shape memory materials, for example nickel-titanium or biocompatible/biodegradable shape memory polymers. The device may employ spring like structures built using a wire or sheets to produce tensile stresses in the intestinal system. In one embodiment, various shapes for the structure may be used to aid in collapsing the device into a smaller diameter so that it can be deployed using an endoscope.

For example, the collapsed device/spring may be compressed into its minimum size using degradable suture to hold it axially in place and subsequently placed in a special tube similar to a catheter with push rod. The device can be delivered into any portion of the intestine using an endoscope and deployed by pushing the device with the push rod into the intestinal tract. Upon deployment of the device into the intestinal tract, the ends of the device engages the interior of the body passage holding it in a specific location and enabling it to transfer stresses to that particular location of the intestine while the suture prevents immediate elongation of the device. After a period of time, the degradable suture dissolves and the structure expands along the longitudinal direction thereby producing longitudinal forces in the growth direction of the intestine. The device is anchored at the end locations by the hydrogel, producing elongation forces on the intestine. The body passage is examined periodically to check the length extension of the portion of the intestines. After a sufficient period, the device can be retracted from the body passage using endoscope or as an option can be left in the body.

An aspect of the disclosure is a mechanical distension apparatus, comprising an elongate, tubular structure configured to be inserted into a body lumen, e.g. intestine, at a treatment location within the lumen. The tubular structure has a central axial channel configured to allow normal operation of said lumen, and first and second ends and being compressible along a longitudinal axis between said ends to form an axially compressed configuration. The tubular structure further includes at least two spaced apart anchor portions configured to engage an internal wall of the lumen at said treatment location while in said axially compressed configuration, wherein the tubular structure is biased to elongate to an expanded configuration, the bias configured to impart a force on the lumen at said treatment location to lengthen the lumen at said location.

Another aspect is a distension system for lengthening a segment of the intestine, comprising the above described tubular structure and an absorbable retaining element configured to retain the tubular structure in its axially compressed configuration, wherein the retaining element is configured to dissolve after a period of time within the lumen to free the tubular structure to impart said force on said lumen.

Another aspect is a method for distending the intestine, comprising the steps of providing the above-described tubular structure to at a treatment location within the organ, compressing said tubular structure along a longitudinal axis of the tubular structure, retaining the tubular structure in an axially compressed state with a dissolvable suture material, inserting the compressed tubular structure into the organ and positioning the tubular structure adjacent the location wherein the hydrogel engages the organ and hold the tubular structure in position; and expanding the tubular structure to an expanded configuration to lengthen the organ at said treatment location.

Spring device. Self-expanding springs have been used to lengthen the small intestine using an intraluminal axial mechanical force, where this biomechanical force stimulates the growth and elongation of the small intestine. Computational models can predict the required mechanical force for any potential patient where this can be advantageous in predicting an individual's tissue response to spring-mediated distraction enterogenesis and can be used toward a safe delivery of the mechanical force.

A method of distraction enterogenesis may use an intraluminal spring where an axial mechanical force is applied within the lumen of the intestine to lengthen the intestine. Mechanical perturbations in the axial direction of the intestinal tract trigger signaling pathways that cause tissue thickening in the radial direction within the distracted segment, as well as adaptive responses in the areas adjacent to the distracted segment.

The spring device may be formed from a memory material, such as of Ni—Ti wire, also known as Nitinol, using a process called “shape setting heat treatment,” during which Ni—Ti wire is wrapped around a mold and heated to 480° C. for 20 minutes followed by rapid cooling. Other memory materials possible are NiTiCu, NiTiPt and a host of other materials including the new ferromagnetic shape memory alloys such as NiMnGa.

Nitinol is a unique material that exhibits a thermally induced crystalline transformation between martensitic phase, a low temperature phase, and austenitic phase, a high temperature phase. The temperature at which martensitic and austenitic phase begin and finish forming are represented by martensite start (Mₛ), martensite finish (M_{f}), austenite start (Aₛ) and austenite finish (A_{f}) temperatures. The austenitic phase of Ni—Ti is a highly ordered body centered cubic structure, B2 phase. In the martensitic phase (i.e., low temperature), it is a monoclinic crystal structure, B19′ phase. The temperature at which these phase change takes place depends on Ni and Ti ratio. A small change in Ni content leads to a large change in its transformation temperatures. For example changing from form Ni₅₂Ti₄₈ to Ni₄₈Ti₅₂ the transformation temperatures change from −100° C. to 120° C. Hence, by changing a specific Ni and Ti content the transformation temperatures can be fixed at a desired operating temperature. The composition is extremely important for applications requiring either shape memory behavior or pseudo elastic behavior.

Pseudo-elasticity is another unique behavior observed in Ni—Ti class of materials. When the material is in austenitic phase, i.e. T>A_{f}, mechanical loading induces an elastic deformation until induced martensitic phase transformation occurs at nearly constant stress. This produces relatively large deformation, up to 10%, due to twin boundary motion and recovers completely upon release of the stress. In other words, a constant force/stress is generated when the material is pre-strained. To observe the pseudo elastic behavior around body temperature, the A_{f} temperature is fixed just below body temperature by varying Ni content. In addition, the material also shows excellent biocompatibility and fatigue resistance.

In some embodiment the spring is a biocompatible nickel-titanium (nitinol) spring, which may be compressed and placed within an absorbable gelatin capsule and then coated with cellulose acetate phthalate, which allows for delayed gelatin capsule degradation.

The term “hydrogel” is used in its conventional sense to refer to a material that absorbs a solvent (e.g. water), undergoes swelling without measurable dissolution, and maintains three-dimensional networks capable of reversible deformation. “Swelling” as referred to herein is meant the isotropic expansion of the hydrogel structure as water molecules diffuse throughout the internal volume of the hydrogel. The properties of copolymer hydrogels disclosed herein may be modulated as desired, by varying the amounts of each component, ratios of each component or the density of specific components, as described in greater detail below. The term hydrogel may include both dessicated and hydrated (e.g., solvent swollen) hydrogels. Of interest are HELP hydrogels.

An Elastin-like Protein (ELP) comprises a recombinant sequence of elastin-like sequences optionally interspersed with cell-adhesive sequences. To engage in crosslinking with chemically modified HA, the ELP is chemically modified to comprise a pendant hydrazine group. Standard bioconjugation chemistry can be used to attach pendant hydrazines at sites of any of lysine, cysteine, or tyrosine amino acids. The optional cell-adhesive sequence within the ELP comprises a motif involved in cell adhesion, which may be selected from an integrin-binding, fibronectin-based, extended RGD sequence, a scrambled RGD sequence, a cell-adhesive sequence derived from collagen type I, e.g. (SEQ ID NO:26) DGEA, a cell adhesive sequence derived from tenascin, e.g. (SEQ ID NO:4) PLAEIDGIELTY, (SEQ ID NO:5) VFDNFVLK, etc.; a cell adhesive sequence derived from laminin, e.g. (SEQ ID NO:6) IKVAV, (SEQ ID NO:7) YIGSR, etc.; a cell adhesive sequence derived from cadherin, e.g. (SEQ ID NO:8) HAVDI, (SEQ ID NO:9) HAVDIHAVDI; and the like.

The cell-adhesive domain of the engineered elastin-like protein can be designed to include alternative peptide-sequences known to interact with cell-surface receptors. These sequences can include peptides derived from native extracellular matrix proteins (e.g. fibronectin, laminin, collagen, tenascin-C) or peptides derived from cell-cell adhesion receptors (e.g. N-cadherin) (Table 1). Selection of the cell adhesive peptide sequence together with the elastin-like region sequence defines the overall hydrophobicity of the engineered protein, and hence controls the lower critical solution temperature (LCST) behavior.

In some embodiments an ELP comprises the structure:

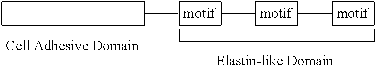

where the cell adhesive domain is from about 15 to about 45 amino acids in length and comprises one or more cell adhesion sequence motifs, which may be selected from RGD, scrambled RGD, no RGD, or any of SEQ ID NO:3 to SEQ ID NO:9. SEQ ID NO:1-3, 10-19 and 22 are exemplary of suitable ELP sequences.

Linker sequences optionally flank the cell adhesion sequence motif, where a peptide linker can be between about 5 to 20, 5 to 15, 5 to 10 or 5 to 9 amino acids in length. Exemplary linkers include linear peptides having at least two amino acid residues such as GG, GAG, GPA, GGGGS (SEQ ID NO:27). Suitable linear peptides include poly glycine, polyserine, polyproline, polyalanine and oligopeptides consisting of alanyl and/or serinyl and/or prolinyl and/or glycyl amino acid residues. In one embodiment a linker comprises the amino acid sequence GSTSGSGKSSEGKG (SEQ ID NO:28), or (GGGGS) n (SEQ ID NO:27), where n is 1, 2, 3, 4, 5, etc.; however many such linkers are known and used in the art and may serve this purpose.

The elastin-like domain is comprised of elastin-like motifs, which include, without limitation, (SEQ ID NO:23) VPGIG; (SEQ ID NO:24) VPGKG; (SEQ ID NO:25) VPGYG. One or more of SEQ ID NO:23, 24 and 25 can be present in a protein. In some embodiments the number of motifs is from 1 to 7, from 1 to 6, from 2 to 5, from 3 to 5; and may be about 5 motifs. Exemplary domain sequences are provided in, for example, SEQ ID NO:20 and 21. Examples include, without limitation, SEQ ID NO:1, LQ(LDASTVYAVGRGDSPASSA[(VPGIG)₂VPGKG(VPGIG)₂]₃)₄ and SEQ ID NO:2, LQ(LDASTVYAVGRDGSPASSA[(VPGIG)₂VPGKG(VPGIG)₂]₃)₄.

The ELP protein is chemically modified to comprise a pendant hydrazine group, and may comprise from about 3 to about 20 hydrazine groups, from about 5 to about 18, from about 10 to about 14 groups. Standard bioconjugation chemistry can be used to attach pendant hydrazines at sites of any of lysine, cysteine, or tyrosine amino acids.

The cell-adhesive sequence concentration of the hydrogel can be varied by adjusting the ratio of ELP comprising an RGD motif, to ELP lacking an RGD motif or comprising scrambled or non-RGD cell-adhesive motifs, as disclosed above. The ratio may be pre-selected for a hydrogel of interest, usually ranging from about 100:0 to 0:100, for example from about 75:25, 50:50, 25:75, 10:90. For some applications, a HELP hydrogel comprises from about 0.25 mM RGD up to about 1.5 mM RGD, e.g. about 0.25 mM, 0.5 mM, 0.75 mM, 1 mM, 1.25 mM, 1.5 mM. Greater than 0.75 mM may be preferred.

TABLE 1
Amino-acid sequences for the bio-active domain
and elastin-like region of HELP gels.

| ECM-derived | | |
|---|---|---|
| Protein | Cell-adhesive domain | Elastin-Like Region |
| h-Fibronectin | (SEQ ID NO: 10) | (SEQ ID NO: 20) |
| | TVYAVTGRGDSPASSAA | (VPGIG)₂(VPGKG)(VPGIG)₂ |
| Laminin (β1 | (SEQ ID NO: 11) | (SEQ ID NO: 20) |
| chain) | VSDPGYIGSRSDDSASASAA | (VPGIG)₂(VPGKG)(VPGIG)₂ |
| Laminin (α1 | (SEQ ID NO: 22) | (SEQ ID NO: 21) |
| chain) | ARKQAASIKVAVSADRASA | (VPGIG)(VPGKG)(VPGYG) |
| | | (VPGIG)(VPGKG)(VPGIG) |
| Collagen I | (SEQ ID NO: 12) | (SEQ ID NO: 20) |
| | VGPAGGDGEAGAQGPP | (VPGIG)₂(VPGKG)(VPGIG)₂ |
| h-Tenascin-C | (SEQ ID NO: 13) | (SEQ ID NO: 20) |
| | SGSGGSGGPLAEIDGIELTYGGSGGSGS | (VPGIG)₂(VPGKG)(VPGIG)₂ |
| h-Tenascin-C | (SEQ ID NO: 14) | (SEQ ID NO: 21) |
| | SGSGGSGGLDVFDNFVLKGGSGGSGS | (VPGIG)(VPGKG)(VPGYG)(VPGIG) |
| | | (VPGKG)(VPGIG) |
| h-Tenascin-C | (SEQ ID NO: 15) | (SEQ ID NO: 20) |
| | SGSGGSGGLDVFDNFVLGGSGGSGS | (VPGIG)₂(VPGKG)(VPGIG)₂ |
| N-cadherin | (SEQ ID NO: 16) | (SEQ ID NO: 20) |
| | SGSGGSGGHAVDIGGSGGSGS | (VPGIG)₂(VPGKG)(VPGIG)₂ |
| N-cadherin | (SEQ ID NO: 17) | (SEQ ID NO: 20) |
| | SGSGGSGGHAVDINGHAVDIGGSGGSGS | (VPGIG)₂(VPGKG)(VPGIG)₂ |
| Non-binding | (SEQ ID NO: 18) | (SEQ ID NO: 20) |
| | SGSGGSGGADHIVGGSGGSGS | (VPGIG)₂(VPGKG)(VPGIG)₂ |
| Non-binding | (SEQ ID NO: 19) | (SEQ ID NO: 20) |
| | TVYAVTGRDGSPASSAA | (VPGIG)₂(VPGKG)(VPGIG)₂ |

Hyaluronic acid is an anionic, non-sulfated glycosaminoglycan distributed widely throughout connective, epithelial, and neural tissues. Hyaluronic acid is a polymer of disaccharides, themselves composed of D-glucuronic acid and N-acetyl-D-glucosamine, linked via alternating β-(1→4) and β-(1→3) glycosidic bonds. Hyaluronic acid can be up to 25,000 disaccharide repeats in length. Polymers of hyaluronic acid can range in size from about 20 kDa to about 1.5 MDa; from about 20 Kda to.

The hyaluronic acid is chemically modified to comprise a pendant benzaldehyde or aldehyde side group. The HA is usually modified at from about 5% to about 30% of the available reactive groups, and may be from about 7% to about 20%, from about 10% to about 15%, and may be around 12% modified.

For an aldehyde functional group the carboxylic acid groups on HA are amidated with propargylamine, generating an HA-alkyne intermediate; then, copper click chemistry was used to react this alkyne with the azide moiety of a heterobifunctional small molecule containing an aldehyde functional group onto the HA, generating HA functionalized with aldehydes.

Benzaldehyde modification can be accomplished by first modifying HA to comprise alkyne groups at a desired concentration, e.g. from about 3% to about 30%. HA-alkynes are then modified with N-(2-azidoethyl)-4-formylbenzamide to generate HA-benzaldehyde.

As used herein, the terms “treatment,” “treating,” and the like, refer to obtaining a desired pharmacologic and/or physiologic effect, such as reduction of viral titer. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease (e.g., reduction in viral titers).

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; avians, and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “determining,” “measuring,” “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of a compound calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

### EXPERIMENTAL

### Example 1

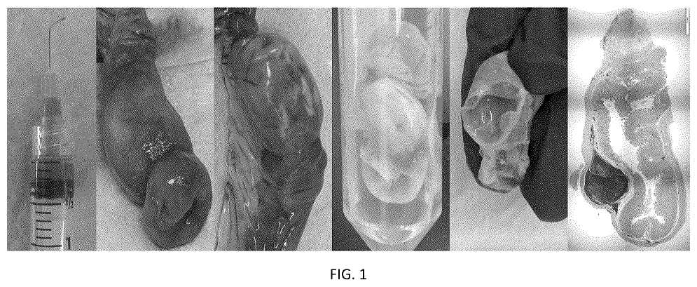

First, we performed an invitro study on a harvested pig bowel to determine the appropriate method and amount of injection. 1.5 to 2 cc of hydrogel was sufficient to cause adequate lumen obstruction to hold the spring in place. The specimen was examined under microscopy to observe the hydrogel, shown in FIG. 1.

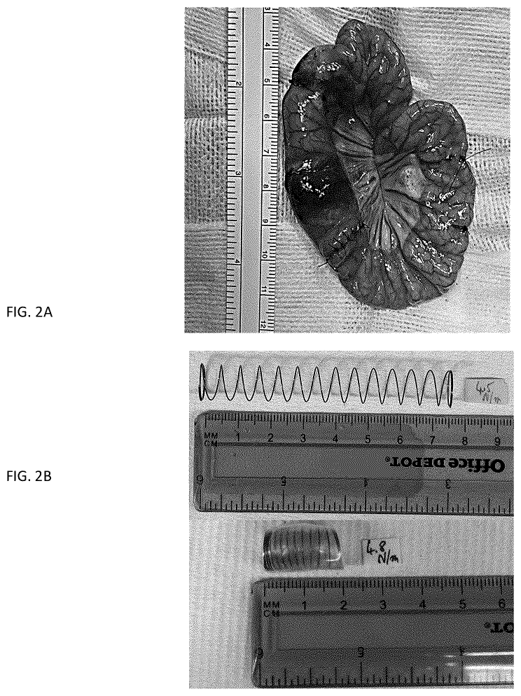

Subsequently, animal study was performed on 6 juvenile pigs, shown in FIG. 2. 1.5 cc of hydrogel was injected into the submucosa of porcine jejunum by everting the transected jejunum. Gelatin encapsulated compressed nitinol spring was placed next to the injected hydrogel. Everted jejunum was unfolded over the spring, and another 1.5 cc of hydrogel was injected distally to keep it in place. Non-absorbable sutures were placed on the serosa of control and spring-containing segments for length comparison. End to end anastomosis was performed to restore continuity. Pigs were placed on CLD and euthanized on POD7. Intestinal segments were evaluated for lengthening between marking sutures and processed for histologic examination. Spring characteristics: Force: 4.4±0.4 N/m Diameter: 1.1±0.1 cm Length: 7.5 cm compressed to 2 cm.

A total of 4 ml of hydrogel was injected into pig intestinal submucosa with 26 gauge needles at the site of a nitinol spring device in 3-4 spots, about 500-650 μl per spot, and repeated on each of the two sides/edges of the spring device. The two components of the hydrogel were mixed together in luer-lock syringes with a syringe connector.

The hydrogel comprised elastin-like protein modified with 14 hydrazines (modification at lysine residues) at a final concentration of 1% w/v; and hyaluronic acid (HA) 60 kDa modified with 12% benzaldehyde (BZA) motifs at a final concentration of 1% w/v. The range of HA bioconjugation with aldehyde was from 6-20%, and the range of HA bioconjugation with benzaldehyde was from 6-30%.

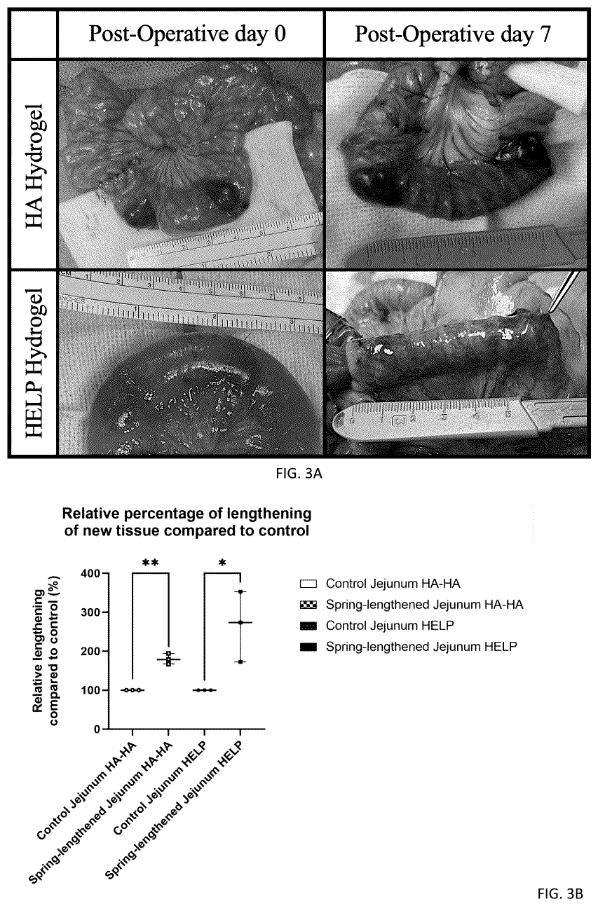

Shown in FIG. 3, although there was some slippage proximally or distally, spring stayed at the injection sites in all animals. The initial length in both spring and control segments was 2.5 cm. control segment shrunk by 72% while the intestinal segment lengthened by 132% in the HA group and 188% in the HELP group which were both statistically significant.

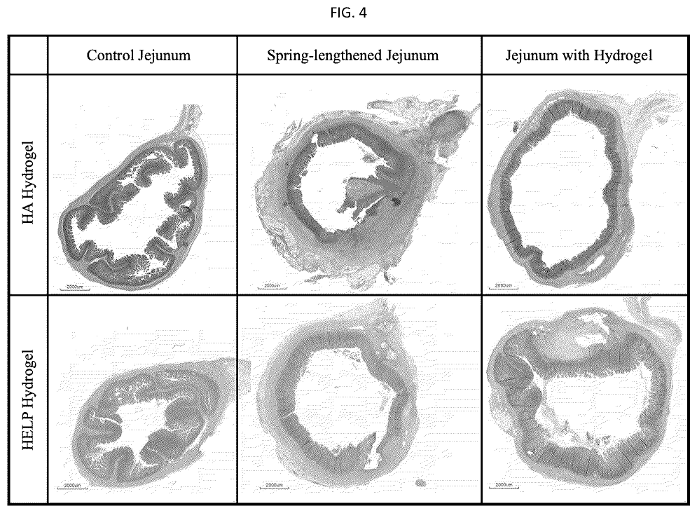

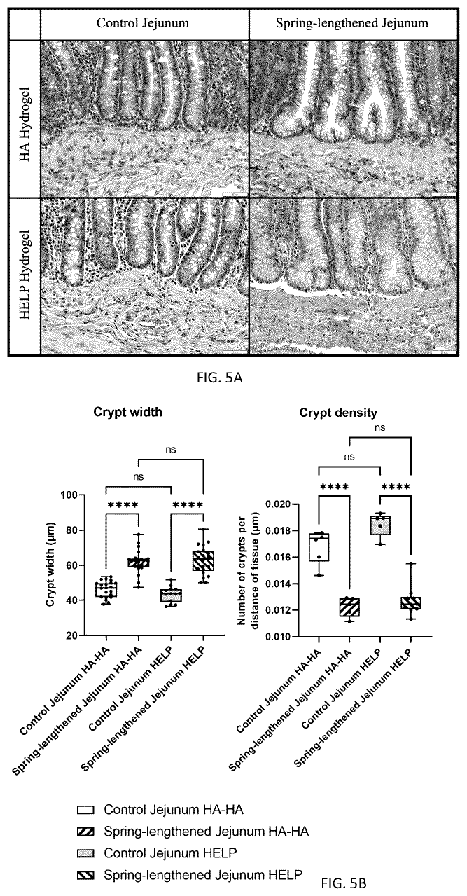

For histologic evaluation, pigs were euthanized, and bowel segments were retrieved. Normal segments and segments of intestine containing the springs were removed and evaluated for lengthening as well as for histologic examination. As shown in FIG. 4, histologic adaptation of the lengthened jejunum was similar among the two groups. We noticed an increase in the crypt width and a decrease in the crypt density of the spring segments compared to the control which might be related to the lengthening process (FIG. 5).

Our observation is consistent with the result of others on type II crypt fission initiated by inflation. Based on this study, mechanical stretch leads to epithelial differentiation and loss of Lgr5 expression. The remaining patches of Stem Cell Zones form multiple new crypts upon collapse.

In conclusion, hydrogel can be used to confine springs within intestinal segments without adverse effects. This is the first experiment toward endoscopic suture-less spring implantation.

### Materials and Methods

ELP-Hydrazine Synthesis. Elastin-Like Protein (ELP) was prepared as described previously. Briefly, ELP sequences were cloned into pET15b plasmids, and a T7 promoter was used to control protein expression. BL21 (DE3) pLysS Escherichia coli (Life Technologies) containing ELP-encoding plasmids were cultured in Terrific Broth to an OD600 of 0.8, and 1 mM isopropyl β-D-1-thiogalactopyranoside (IPTG) was used to induce expression. Bacteria were allowed to express protein for 7 h, and were subsequently harvested by centrifugation, suspended in TEN buffer (10 mM Tris, 1 mM EDTA, and 100 mM NaCl, pH 8.0), and lysed via three cycles of freeze-thaw. Cell lysate was treated with deoxyribonuclease (DNase) and 1 mM phenylmethanesulfonyl fluoride (PMSF) to inhibit proteolysis. ELP was purified by an alternating sequence of centrifugation steps at 4° C. and 37° C., followed by dialysis against deionized water for 4 shifts (48 h, 4 L volume per shift), then frozen at −80° C. and lyophilized. To modify ELP amines with hydrazine functional groups, lyophilized ELP (210 mg) was completely dissolved at 7 wt % in 3 mL of anhydrous dimethyl sulfoxide (DMSO) and then diluted to 3.5 wt % with 3 mL of anhydrous N,N-dimethylformamide (DMF). In a round-bottom flask, 3 mL of anhydrous DMF was used to separately dissolve tri-Boc-hydrazinoacetic acid (2 equiv:ELP amine), hexafluorophosphate azabenzotriazole tetramethyl uronium (HATU, 2 equiv:ELP amine), and 4-methylmorpholine (4.5 equiv:ELP amine), and this vessel was stirred for 5 min to allow HATU to activate the free acids on the tri-Boc-hydrazinoacetic acid. Next, the ELP solution was added to the round-bottom flask dropwise with stirring. The reaction was allowed to proceed overnight at room temperature (RT). The product was precipitated in ice-cold ether, centrifuged, and dried, yielding the Boc-protected ELP-hydrazine intermediate. This intermediate was analyzed by 1H NMR to quantify the modification. Modification efficiency was determined by comparing the integrated signal of the Boc protons (δ 1.5-1.35) to the aromatic protons of tyrosine residues on ELP (δ 7.00 and 6.62). To remove the Boc protecting groups, the ELP-hydrazine intermediate was dissolved at 2 wt % in 1:1 DCM:TFA with 2.5% v/v triisopropylsilane and stirred at RT for 4 h in a vented round-bottom flask. The product was precipitated in ether, centrifuged, and dried, then dissolved in DI water and dialyzed against DI water for 3 shifts (24 h, 4 L volume per shift), and lyophilized.

Hyaluronic Acid Modification. 100 kDa sodium hyaluronate (HA, Lifecore Biomedical, Chaska, MN, USA) was modified to have an aldehyde functional group by the following overall procedure: first the carboxylic acid groups on HA were amidated with propargylamine, generating an HA-alkyne intermediate; then, copper click chemistry was used to react this alkyne with the azide moiety of a heterobifunctional small molecule containing an aldehyde functional group onto the HA, generating HA functionalized with aldehydes.

HA-alkyne of 12% modification: HA was dissolved in 2-(N-morpholino) ethanesulfonic acid (MES) buffer (0.2 M, pH 4.5) to a concentration of 10 mg/mL. To this solution, N-hydroxysuccinimide (NHS, 0.8 eq. to the HA dimer unit), 1-ethyl-3-(3-dimethylaminopropyl) carbodiimide (EDC, 0.8 eq.), and propargyl amine (0.8 eq.) were added successively. After adjusting pH to 6, the mixture was stirred at RT for 4 h. The solution was then dialyzed against DI water for 6 shifts (3 d, 4 L volume per shift) and lyophilized to give a white powder.

HA-alkyne of 30% modification: Sodium hyaluronate was dissolved in MES buffer (0.2 M, pH 4.5) to a concentration of 10 mg/mL. To this solution, NHS (1.5 eq. to the HA dimer unit), EDC (1.5 eq.), and propargyl amine (1.0 eq.) were added successively. After adjusting pH to 6, the mixture was stirred at RT for 4 h. The solution was then dialyzed against DI water for 6 shifts (3 d, 4 L volume per shift) and lyophilized to give a white powder.

HA-alkynes were then modified by the following small molecule, 1, to generate HA-benzaldehyde. The small molecule was generated as follows:

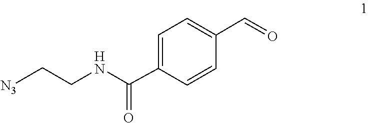 [SMILES: [N-]=[N+]=NCCNC(=O)c1ccc(C=O)cc1]

N-(2-azidoethyl)-4-formylbenzamide (1) was synthesized according to the method published in Biomaterials, 2018, 154, 213-222: HA was modified with molecule 1 according to previously reported procedure with minor modifications. HA-alkyne (300 mg) was dissolved in PBS at 2 wt % followed by the addition of 1 (1 eq. to HA dimer unit). A minimal amount of DMSO was used to dissolve 1 before it was added to the HA solution. The solution was then bubbled with N₂ for 30 min. Copper (II) sulfate pentahydrate (0.004 eq.) and sodium ascorbate (0.06 eq.) were dissolved in DI water, bubbled with N₂, and added to the HA solution. After stirring at RT for 1 d, the mixture was dialyzed against DI water for 3 d and lyophilized. Since the proton signals of aromatic rings on the benzaldeyde moiety overlap with triazole groups, the degree of modification on HA-benzaldehyde was quantified by integration of the proton signal (δ=7.5−8, 5H) relative to that of the methyl groups on N-acetylglucosamine of HA backbone (δ=1.8, 3H).

HA-aldehyde synthesis: HA-aldehyde was synthesized according to the method published in Biomaterials, 2009, 30, 2499-506: HA was first dissolved at 0.4 w/v % in Milli-Q water while stirring at room temperature. An aqueous solution of 0.1 M sodium periodate was added dropwise, and the reaction was stirred overnight at room temperature in the dark. The following day, ethylene glycol was added for 1 hr to inactivate any unreacted periodate. The solution was then purified by dialysis with a 10,000 MWCO membrane against Milli-Q water for 3 days, with fresh water changed in shifts of 12 hours. After dialysis, the dry product was obtained via freeze-drying.

Polyethylene glycol-benzaldehyde (PEG-BZA) synthesis: PEG-BZA was synthesized as previously described. Briefly, 4-formyl benzoic acid (0.528 g, 3.52 mmol, 2.1 eq. per amine; Sigma) was dissolved in 5 mL anhydrous dimethylformamide (DMF; Sigma) and activated with HATU (1.216 g, 3.2 mmol, 2 eq.; Sigma) and 4-methylmorpholine (0.792 mL, 7.2 mmol, 4.5 eq.; Sigma). The reaction was allowed to stir for 5 minutes before the addition of 4-arm 10 kDa PEG-amine (4 g, 0.2 mmol; Creative PEGworks) dissolved in 5 mL DMF for a total reaction volume of 10 mL. The reaction was allowed stir at room temperature overnight. The final polymer was precipitated in ethyl ether (Thermo Fisher), pelleted by centrifugation at 22,000 rcf for 20 minutes, and re-dissolved in Milli-Q water. PEG-BZA was dialyzed (MWCO: 3,500 Da; Spectrum) against Milli-Q water for 3 days at 4° C., and dialysis water was changed 2-3 times per day. PEG-BZA was lyophilized and stored at −20° C. Modification of PEG-BZA was estimated using 1H-NMR (500 MHz). PEG-BZA was dissolved in deuterated water (D₂O; Sigma) at 10 mg/mL. δ=9.9 ppm (1H, s, aldehyde); δ=7.93 and 7.82 ppm (2H each; d; benzene ring); δ=3.56 (217H per arm; s; PEG).

To form HELP hydrogels, ELP and HA gel components were separately dissolved to form 2 w/v % stock solutions in PBS. A selected modified HA component was added to equal volume of the ELP solution. The pipette tip was then used to mix the two hydrogel. Hydrogels may be allowed to crosslink for 10 min at room temperature.

The invention claimed is:

1. A method of anchoring a device or therapeutic entity to a segment of the intestine in an individual, the method comprising:
injecting into the intestinal wall a dose of hydrogel sufficient to narrow the intestinal lumen, wherein the hydrogel comprises:
a first component comprising a defined ratio of hyaluronic acid (HA) modified to comprise a pendant reactant group aldehyde and/or HA modified to comprise a pendant reactant group benzaldehyde; and
a second component comprising an elastin-like protein (ELP) modified to comprise a pendant reactant group hydrazine group; wherein cross-links between the first component and the second component are formed to generate a hydrogel upon mixing;
wherein the narrowing of the intestinal lumen anchors the device or therapeutic entity with the segment of the intestine.

2. The method of claim 1, wherein the device or therapeutic entity is an in situ expander for lengthening the intestine.

3. The method of claim 2, wherein the in situ expander is a compressed coiled spring.

4. The method of claim 1, wherein the individual suffers from short bowel syndrome.

5. The method of claim 1, wherein the ratio of hyaluronic acid modified to comprise a pendant aldehyde, and hyaluronic acid modified to comprise a pendant benzaldehyde is from 75:25 to about 25:75.

6. The method of claim 1, wherein the ELP comprises from 1 to 7 elastin-like motifs.

7. The system of claim 1, wherein the ELP comprises from 3 to 20 hydrazine groups.

8. The method of claim 1, wherein the hydrogel is extruded though a syringe or catheter.
